(19) United States
(12) Patent Application Publication
(10) Pub. No.: US 2007/0178520 A1
(43) Pub. Date: Aug. 2, 2007

(54) Screening assay for modulators of interaction between interleukin-12 and/or -23 with their receptors

(76) Inventor: Barbara Wolff-Winiski, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/590,400

(22) PCT Filed: Feb. 25, 2005
(86) PCT No.: PCT/EP05/02015

§ 371 Date: Aug. 23, 2006

Related U.S. Application Data
(60) Provisional application No. 60/548,581, filed on Feb. 27, 2004.
(60) Provisional application No. 60/605,772, filed on Aug. 31, 2004.

(51) Int. Cl. G01N 33/53

(52) U.S. Cl. 435/7.1

(57) ABSTRACT

The invention relates to a screening assay, e.g. to an assay for identifying an agent that modulates the interaction of interleukin-23 and/or interleukin-12 with a corresponding receptor.

The present invention relates to a screening assay, e.g. to an assay for identifying an agent that modulates the interaction of interleukin-23 and/or interleukin-12 with a corresponding receptor thereof.

It is known from the literature that interleukin-23 and interleukin-12 play an important role as mediators, e.g. in the immune system, see e.g. Puccetti P. et al., Crit. Rev. Immunol. 2002, 22 (5-6), 373-90, in infectious diseases, see e.g. Holscher C. et al, J. Immunol. 2001, 167(12)6957-66 and in inflammation, see e.g. Lupusoru C. E. et al., Rev. Med. Chir. Soc. Med. Nat. Iasi, 2002, 106(1), 24-9.

In one aspect the present invention provides an assay for identifying an agent that modulates the interaction of interleukin-23 and/or interleukin-12 with a corresponding receptor thereof comprising
a) contacting interleukin-23 and/or interleukin-12 with a corresponding interleukin receptor in the absence and in the presence of a candidate compound which is expected to modulate the interaction of said interleukin with said receptor for a sufficient period of time so that a complex between said interleukin and said receptor can be formed,
b) optionally separating the complex from uncomplexed fractions,
c) detecting the complex formed in step a),
d) determining whether there is a difference in the amount of complex formed in case a candidate compound was absent or present in step a), and
e) choosing a candidate compound where a difference is determined in step d) as an agent,
   e.g. the receptor is the interleukin-23 p19 receptor and/or the interleukin-12 p40 receptor, e.g. a receptor as described by Parham Ch. et al., Journal of Immunology, 2002, 168:5699-5708.

lnterleukin-23 and interleukin-12 as used in the present invention include full length proteins, e.g. wild type proteins, or a part thereof. “(A) part thereof” as used herein is understood to be an interleukin-23 or an interleukin-12, which retains specificity for an interleukin-23 receptor or for an interleukin-12 receptor. E. g. the interleukin-23 or interleukin-12 is a protein, which is smaller than the wild type protein, e.g. a protein having less amino acids than the wild type protein, or a protein having a modification, e.g. a mutation, e.h. having a substitution or an addition of an amino acid as compared to the wild type protein, but still retaining its specificity for the corresponding receptor.

Interleukin-23 and interleukin-12 may be from human or animal origin, prefereably human origin. It may be obtained from natural sources or by using recombinant or chemical techniques according, e.g. analogously, to procedures as conventional.

Interleukin-23 and interleukin-12 as defined herein are also designated as “interleukin(s) of the present invention”.

A receptor of the present invention includes a wild-type receptor for interleukin-23, interleukin 12 or a part thereof. “A part thereof” as used herein is understood to be a modified or mutated interleukin-23 and interleukin-12 receptor, which retains its specificity for the corresponding interleukin. E.g. the receptor is a molecule, such as a protein, which is smaller than the wild type receptor, e.g. a receptor protein having less amino acids than the wild type receptor protein, or a molecule having a modification, e.g. a mutation, such as a molecule having a substitution or an addition of a nucleotide or an amino acid as compared to the wild type receptor, but still retaining its specificity for the corresponding interleukin. The receptor may be isolated from natural sources or can be obtained by using recombinant or chemical techniques according, e.g. analogously, to procedures as conventional. The receptors as defined herein are also designated as “receptors of the present invention”.

In another aspect the present invention provides an assay for identifying an agent that modulates the interaction of interleukin-23 and/or interleukin-12 with a corresponding receptor, wherein the receptor is fused to an immunoglobulin or a fragment thereof.

An immunoglobulin (Ig) as used in the present invention is understood to be any kind of immunoglobulin, e.g. IgA, IgG, IgM, preferably IgG. “A fragment” of an immunoglobulin includes any known immunoglobulin fragments, e.g. a Fab part of an Ig, such as a Fab part of IgG. Preferably the receptor-immunoglobulin fusion protein is an interleukin-23 receptor/Fc fusion protein or an interleukin-12 β1/Fc fusion protein.

The receptors fused to an immunoglobulin as defined herein are also designated “fusion proteins of the present invention”.

Optionally a complex formed can be separated from uncomplexed fractions.

In case the complex formation reaction is carried out as a homogenous reaction in solution, the separation can be carried out according, e.g. analogously, to methods as conventional, e.g. chromatographically, e.g. size exclusion chromatography.

In case the complex formation reaction is carried out as a heterogenic reaction, e.g. on a solid phase, the complex can be separated according, e.g. analogously, to methods as conventional, e.g. by washing the solid phase to which the complex formed is bound, e.g. by use of appropriate washing solutions.

For detecting the complex formed detection means may be used. Such detection means include those as conventional in the field of assays, e.g. immunoassays, such as enzyme linked immunoassays (ELISAs). Detection means used in the present invention comprise molecules which recognize interleukin-23 and/or interleukin-12, e.g. a molecule which is directly or indirectly detectable. Detection means of the present invention preferably comprise an antibody, e.g. an antibody which recognizes interleukin23 and/or interleukin-12, e.g. a label bearing interleukin-12 antibody.

The label may be one as conventional, e.g. biotin or an enzyme such as alkaline phosphatase (AP), horse radish peroxidase (HRP) or peroxidase (POD) or a fluorescent molecule, e.g. a fluorescent dye. Preferably the label is biotin. The label bearing molecule, e.g. the label bearing antibody, may be detected according to methods as conventional, e.g. via fluorescence measurement or enzyme detection methods.

Optionally the interleukin, the receptor or the fusion protein of the present invention or the detectable molecule comprised in the detection means is immobilized on a solid phase. An appropriate solid phase includes e.g. one as conventional, e.g. a plastic plate like a polystyrene or polyvinyl plate, especially a microtiter plate. Also microbeads can be used as a solid phase, e.g. coated microbeads. The solid phase can be coated with a coating material the nature of which depends e.g. on the label comprised in the detection means. The coating material should be able to bind to the label, e.g. the label is biotin and the coating material includes streptavidin, e.g. covalently bound to the solid phase.

In a preferred aspect the interleukin receptor, e.g. the fusion protein of the present invention, is immobilized on a solid phase, e.g. on microtiter plates. The complex formed on the solid phase, e.g. on microtiter plates, may be detected with detection means comprising a biotin-labeled anti-interleukin-12 antibody, strepatvidin-alkaline phosphatase and a phosphatase substrate and measuring the absorbance at a defined wavelength, e.g. at 405 nm.

A candidate compound includes compound(s)(libraries) from which its modulating effect on the interaction of interleukin-23 and/or interleukin-12 with a corresponding receptor thereof can be determined. Compound (libraries) include for example oligopeptides, polypeptides, proteins, antibodies, mimetics, small molecules, e.g. low molecular weight compounds (LMW's).

An agent is a compound which influences (inhibits) the binding of interleukin-23 and/or interleukin-12 to a corresponding receptor thereof as determined, e.g. detected, in step d) in an assay provided by the present invention.

An agent is one of the chosen candidate compounds and may include oligopeptides, polypeptides, proteins, antibodies, mimetics, small molecules, e.g. low molecular weight compounds (LMW's). An agent includes one or more agents, e.g. a combination of agents.

In another aspect the present invention provides an assay for identifying an agent that modulates the interaction of interleukin-23 with a corresponding receptor thereof comprising
a) contacting interleukin-23 with the interleukin-23 p19 receptor and/or the interleukin-12 p40 receptor in the absence and in the presence of a candidate compound which is expected to modulate the interaction of said interleukin with said receptor for a sufficient period of time so that a complex between said interleukin and said receptor can be formed,
b) optionally separating the complex form uncomplexed fractions,
c) detecting the amount of complex formed in step a),
d) determining whether there is a difference in the amount of complex formed in case a candidate compound was absent or present in step a), and
e) choosing a candidate compound where a difference is determined in step d) as an agent,
   e.g. the detection means for detecting a complex formed between interleukin-23 and the interleukin-23 p19 receptor and/or the interleukin-12 p40 receptor comprises a label bearing, e.g. biotinylated, interleukin-12 antibody.

In another aspect the present invention provides an assay for identifying an agent that modulates the interaction of interleukin-12 with a corresponding receptor thereof comprising
a) contacting interleukin-12 with the interleukin-12 p40 receptor in the absence and in the presence of a candidate compound which is expected to modulate the interaction of said interleukin with said receptor for a sufficient period of time so that a complex between said interleukin and said receptor can be formed,
b) optionally separating the complex form uncomplexed fractions,
c) detecting the complex formed in step a),
d) determining whether there is a difference in the amount of complex formed in case a candidate compound was absent or present in step a), and
e) choosing a candidate compound where a difference is determined in step d) as an agent,
   e.g. the detection means for detecting a complex formed between interleukin-12 and the interleukin-12 p40 receptor comprises a label bearing, e.g. biotinylated, interleukin-12 antibody.

In another aspect the present invention provides a kit for identifying an agent that modulates the interaction of interleukin-23 and/or interleukin-12 with a corresponding receptor comprising
a) interleukin-23 and/or interleukin-12,
b) the interleukin-23 p19 receptor and/or the interleukin-12 p40 receptor,
c) optionally detection means,
d) instructions for use of said kit, and
e) optionally a solid phase.

In another aspect the present invention provides a kit as provided by the present invention, wherein

said detection means comprise a label bearing, e.g. biotinylated, interleukin-12 antibody,

the interleukin receptor is fused to an immunoglobulin or a fragment thereof, e.g. an interleukin-23 receptor/Fc fusion protein or an interleukin-12 receptor β1/Fc fusion protein.

In another aspect the present invention provides a kit for identifying an agent that modulates the interaction of interleukin-23 with a corresponding receptor comprising
a) interleukin-23,
b) the interleukin-23 p19 receptor and/or the interleukin-12 p40 receptor,
c) optionally detection means,
d) instructions for use of said kit, and
e) optionally a solid phase.

In another aspect the present invention provides a kit for identifying an agent that modulates the interaction of interleukin-12 with a corresponding receptor comprising
a) interleukin-12,
b) the interleukin-12 p40 receptor,
c) optionally detection means,
d) instructions for use of said kit, and
e) optionally a solid phase.

Such kit as provided by the present invention may further comprise a substantial component including an appropriate environment of a sample to be tested and, e.g. appropriate means to determine the effect of a candidate compound in a sample to be tested.

In another aspect the present invention provides an agent identified by an assay of the present invention.

In another aspect the present invention provides the use of an agent of the present invention as a pharmaceutical.

In another aspect the present invention provides the use of an agent of the present invention for the manufacture of a medicament for the treatment of a disease selected from the group consisting of autoimmune related diseases, including allergic diseases, inflammatory diseases and infectious diseases.

In another aspect the present invention provides a pharmaceutical composition comprising an agent of the present invention beside at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such compositions may be manufactured according, e.g. analogously to a method as conventional, e.g. by mixing, granulating, coating, dissolving or lyophilizing processes. Unit dosage forms may contain, for example, from about 0.5 mg to about 1000 mg, such as 1 mg to about 500 mg.

In another aspect the present invention provides the use of the interleukin-23 p19 receptor and interleukin-12 p40 receptor for identifying an agent that modulates the interaction of interleukin-23 with one of said receptors or parts thereof.

In another aspect the present invention provides the use of the interleukin-12 p40 receptor for identifying an agent that modulates the interaction of interleukin-12 with said receptor.

In another aspect the present invention provides a method for determining whether a receptor is specific for interleukin-23 or interleukin-12 or both or none, comprising
a) providing a receptor,
b) contacting interleukin-23 with the receptor of step a) for a sufficient period of time so that a complex between said interleukin and said receptor can be formed,
c) contacting interleukin-12 with the receptor of step a) for a sufficient period of time so that a complex between said interleukin and said receptor can be formed,
d) optionally separating the complex formed in step b) and/or
c) from uncomplexed fractions,
e) detecting the complex formed in step b) and/or in step c) with detection means,
f) determining whether the receptor is
   specific for interleukin-23, which is the case if a complex formation of step b) and no complex formation of step c) is detected, or
   specific for interleukin-12, which is the case if a complex formation of step c) and no complex formation of step b) is detected, or
   specific for both interleukin-23 and interleukin-12, which is the case if a complex formation of step b), and a complex formation of step c) is detected, or
   unspecific for interleukin-23 and interleukin-12, which is the case if no complex formation of step b), and no complex formation of step c) is detected.

### DESCRIPTION OF THE FIGURES

FIG. 1 shows the concentration dependent binding curve of interleukin-23 to the interleukin-23 p19 receptor, wherein the complex formed is detected with detection means comprising a biotinylated anti-interleukin-12 antibody, avidin and alkaline phosphatase substrate reagent. The absorbance at 405 nm (OD₄₀₅) is measured.

FIG. 2 shows the concentration dependent binding curve of interleukin-23 to the interleukin-12 receptor β1, wherein the complex formed is detected with detection means comprising a biotinylated anti-interleukin-12 antibody, avidin and alkaline phosphatase substrate reagent. The absorbance at 405 nm (OD₄₀₅) is measured.

In the following examples all temperatures are in degree centigrade and are uncorrected.

The following ABBREVIATIONS are used:

| | |
|---|---|
| BSA | bovine serum albumin |
| Fc | constant region of immunoglobulin G |
| PBS | phosphate buffered saline |
| RT | room temperature |

### EXAMPLES

### Example 1

### IL-23 Receptor Binding Assay

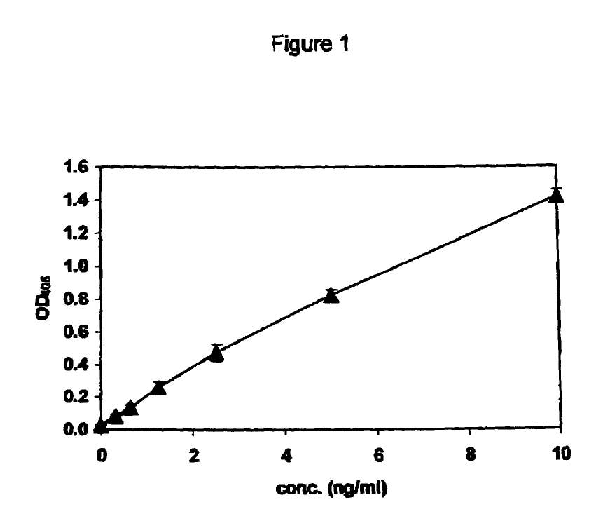

An IL-23 p19 receptor/Fc fusion protein (R&D Systems #1400-IR9) is coated onto 96-well plates (Nunc Maxisorb #442404) at a concentration of 1 μg/ml in PBS, pH 7.4, 100 μl/well. All incubation steps are carried out at RT in a humidified chamber overnight. The plates are emtied and filled with 200 μl/well of SuperBlock (Pierce #37535). After 1 hour, the blocking reagent is discarded. 100 μl/well of IL-23 (R&D Systems #1290-IL) are added in triplicate at different concentrations in assay diluent comprising 20 mM Tris-HCl, 150 mM NaCl, 0.1 % of BSA, 0.05% of Tween 20 in PBS, pH 7.4. for 1.5 hours. The plates are washed 4 times with wash buffer (0.05% Tween 20 in PBS, pH7.4). 100 μl/well of a biotinylated goat anti-IL-12 antibody (R&D Systems #BAF219) at a concentration of 250 ng/ml in assay buffer are added for 1.5 hours. After washing 4 times with wash buffer, the plates are incubated with 50 μl/well of ExtraAvidin (Sigma #E-2636) diluted 1:2000 in SuperBlock. After 1.5 hours, the plates re-washed 4 times with wash buffer and 100 μl/well of alkaline phosphatase substrate reagent (BioRad #172-1063) are added. Color development is stopped by addition of 50 μl/well of 2 N NaOH. The absorbance is read on a SLT microtiter plate reader at 405 nm with a reference wavelength of 690 nm. Results are shown in FIG. 1.

### Example 2

### IL-12 Receptor β1 Binding Assay

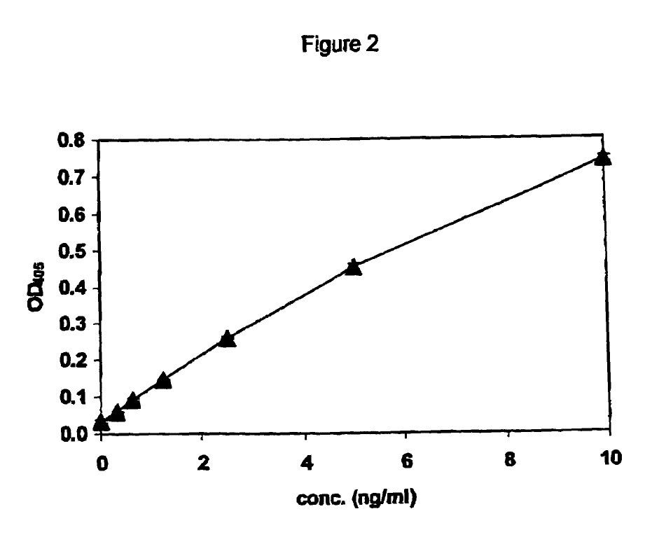

The assay is carried out as described in example 1 but using the IL-12 receptor β1/Fc fusion protein (R&D Systems #839-B1). Results are shown in FIG. 2.

1. Assay for identifying an agent that modulates the interaction of interleukin-23 and/or interleukin-12 with a corresponding receptor thereof comprising
a) contacting interleukin-23 and/ or interleukin-12 with a corresponding interleukin receptor in the absence and in the presence of a candidate compound which is expected to modulate the interaction of said interleukin with said receptor for a sufficient period of time so that a complex between said interleukin and said receptor can be formed,
b) optionally separating the complex from uncomplexed fractions,
c) detecting the complex formed in step a),
d) determining whether there is a difference in the amount of complex formed in case a candidate compound was absent or present in step a), and
e) choosing a candidate compound where a difference is determined in step d) as an agent.

2. The assay of claim 1, wherein the receptor is the interleukin-23 p19 receptor and/or the interleukin-12 p40 receptor.

3. The assay of claim 1, wherein the receptor is fused to an immunoglobulin or a fragment thereof.

4. The assay of claim 1, wherein
the interleukin is interleukin-23,
the receptor is the interleukin-23 p19 receptor and/or the interleukin-12 p40 receptor.

5. Assay of claim 1, wherein
the interleukin is interleukin-12,
the receptor is the interleukin-12 p40 receptor.

6. Kit for identifying an agent that modulates the interaction of interleukin-23 and/or interleukin-12 with a corresponding receptor comprising
a) interleukin-23 and/or interleukin-12,
b) the interleukin-23 p19 receptor and/or the interleukin-12 p40 receptor,
c) optionally detection means,
d) instructions for use of said kit, and
e) optionally a solid phase.

7. The kit of claim 6, wherein said detection means comprise a label bearing interleukin-12 antibody.

8. The kit of claim 6, wherein the interleukin receptor is fused to an immunoglobulin or a fragment thereof.

9. An agent identified by an assay of claim 1.

10. (canceled)

11. (canceled)

12. Pharmaceutical composition comprising an agent of claim 9 and at least one pharmaceutical excipient.

13. (canceled)

14. Method for determining whether a receptor is specific for interleukin-23 or interleukin-12 or both or none, comprising
a) providing a receptor,
b) contacting interleukin-23 with the receptor of step a) for a sufficient period of time so that a complex between said interleukin and said receptor can be formed,
c) contacting interleukin-12 with the receptor of step a) for a sufficient period of time so that a complex between said interleukin and said receptor can be formed,
d) optionally separating the complex formed in step b) and/or c) from uncomplexed fractions,
e) detecting the complex formed in step b) and/or in step c) with detection means,
f) determining whether the receptor is
specific for interleukin-23, which is the case if a complex formation of step b) and no complex formation of step c) is detected, or
specific for interleukin-12, which is the case if a complex formation of step c) and no complex formation of step b) is detected, or
specific for both interleukin-23 and interleukin-12, which is the case if a complex formation of step b), and a complex formation of step c) is detected, or
unspecific for interleukin-23 and interleukin-12, which is the case if no complex formation of step b), and no complex formation of step c) is detected.
